(19) United States
(12) Patent Application Publication
(10) Pub. No.: US 2026/0063608 A1
(43) Pub. Date: Mar. 5, 2026

(54) MACHINE LEARNING GUIDED ELECTROCHEMICAL IMPEDANCE SPECTROSCOPY FOR CALIBRATION-FREE PHARMACEUTICAL MOISTURE CONTENT MONITORING

(71) Applicant: Purdue Research Foundation, West Lafayette, IN (US)

(72) Inventors: Na Lu, West Lafayette, IN (US); Yining Feng, West Lafayette, IN (US); Guangshuai Han, West Lafayette, IN (US)

(21) Appl. No.: 19/311,264

(22) Filed: Aug. 27, 2025

Related U.S. Application Data
(60) Provisional application No. 63/689,611, filed on Aug. 30, 2024.

(51) Int. Cl. G01N 33/15; G01R 27/26; G16C 20/70

(52) U.S. Cl. CPC G01N 33/15; G01R 27/2682; G16C 20/70

(57) ABSTRACT

The invention provides systems and methods for machine learning guided electrochemical impedance spectroscopy for calibration-free pharmaceutical moisture content monitoring. In certain aspects, the invention provides a system for determining moisture content in a sample that includes an electrochemical impedance spectroscopy (EIS) apparatus; and a processor configured to: receive electrical properties of a sample from the EIS apparatus; and correlate the electrical properties to moisture content of the sample.

### RELATED APPLICATION

The present application claims the benefit of and priority to U.S. provisional patent application Ser. No. 63/689,611, filed Aug. 30, 2024, the content of which is incorporated by reference herein in its entirety.

### GOVERNMENT SUPPORT

This invention was made with government support under IIP-1916691 awarded by the National Science Foundation. The government has certain rights in the invention.

### FIELD OF THE INVENTION

The invention generally relates to systems and methods for machine learning guided electrochemical impedance spectroscopy for calibration-free pharmaceutical moisture content monitoring.

### BACKGROUND

Critical to the pharmaceutical industry, precise moisture monitoring in raw materials is not just a quality concern; it's a cornerstone of patient safety and manufacturing excellence. Balancing this delicate equation not only safeguards health but also streamlines production, yielding significant cost efficiencies. Therefore, monitoring the raw materials of pharmaceutical drugs is a crucial step in the production process. However, the methods available for real-time moisture content monitoring are highly limited. Common techniques for moisture content detection possess various limitations, making in-line water content monitoring extremely challenging. For instance, near-infrared (NIR) spectroscopy requires calibration for different materials, which adds complexity and time-consuming steps to the process. Moreover, NIR measurements are not sufficiently accurate in determining water content. Karl Fischer titration, while accurate for detecting water content in pharmaceuticals, poses a significant time requirement and is unsuitable for implementation on a production line. As a result, much of the research focuses on streamlining the testing process to reduce time consumption.

### SUMMARY

The invention recognizes that moisture content of samples, such as pharmaceutical powders, can significantly impact the physical and chemical properties of final products, such as but not limited to drug formulations, solubility, flowability, and stability. However, current technologies (i.e. Karl Fischer titration, NIR spectroscopy) for measuring moisture content in materials, such as pharmaceutical materials, requires extensive calibration process for each new type of material, leading to poor consistency and lack of speed.

To address this challenge, the invention generally relates to using impedance spectroscopy to enable accurate, rapid testing moisture content of pharmaceutical materials with minimal to zero calibration. By utilizing electrochemical impedance spectroscopy (EIS) signals, a strong correlation is established between electrical properties and varying moisture contents in samples, such as but not limited to pharmaceutical samples. In certain embodiments, equivalent circuit modeling is employed to unravel the underlying mechanism, providing valuable insights into the sensitivity of impedance spectroscopy to moisture content variations. Furthermore, certain embodiments incorporate AI techniques, utilizing a 1D Convolutional Neural Network (1DCNN) model to effectively process the complex spectroscopy data. The proposed model integrates frequency spectrum information, equivalent circuit indices, and material characteristics to achieve exceptional predictive accuracy, with an average error of only 1.06% in moisture content estimation. The systems and methods of the invention offer a prompt and reliable means for real-time moisture content monitoring, with applications extending beyond pharmaceuticals to food, energy, environmental, and healthcare contexts.

In certain aspects, the invention provides systems for determining moisture content in a sample, the system comprising: an electrochemical impedance spectroscopy (EIS) apparatus; and a processor configured to: receive electrical properties of a sample from the EIS apparatus; and correlate the electrical properties to moisture content of the sample. In other aspects, the invention provides methods for determining moisture content in a sample, the system involving receiving to a processor electrical properties of a sample from an electrochemical impedance spectroscopy (EIS) apparatus; and correlating via the processor the electrical properties to moisture content of the sample to thereby determine moisture content in the sample. In certain embodiments of the systems and methods herein, the processor is integrated into the EIS apparatus. In certain embodiments of the systems and methods herein, the processor is remotely coupled to the EIS apparatus.

In certain embodiments of the systems and methods herein, the correlate step occurs without calibration. In certain embodiments of the systems and methods herein, moisture content is determined in real-time.

In certain embodiments of the systems and methods herein, the correlate step utilizes a statistical analysis approach that comprises: obtaining moisture content data of a sample; obtaining simultaneously electrical response signals of the sample; and assessing variations in the electrical response signals at different moisture levels compared to electrical response signals obtained under dry conditions for the sample.

In certain embodiments of the systems and methods herein, the correlate step utilizes equivalent circuit modeling calculates EIS indices. In certain embodiments of the systems and methods herein, the EIS indices are utilized to establish a correlation with the moisture content of the sample. In certain embodiments of the systems and methods herein, the equivalent circuit modeling comprises: receive input signals from the sample, which are provided through two channels: a first channel containing the real-time moisture content signal of the sample and a second channel containing baseline signal of the sample under dry conditions; apply 1D convolutional layers, which learn and extract features from the input signals; apply pooling layers to reduce dimensionality and highlight salient features of the input signals; pass through a flatten layer; and combine with non-signal descriptors in a Multi-Layer Perceptron (MLP) layer.

In certain embodiments of the systems and methods herein, the sample is a pharmaceutical sample.

### BRIEF DESCRIPTION OF THE DRAWINGS

FIG. 1 shows an experimental setup for measurement of electrical signal.

FIG. 2 shows a schematic of the 1DCNN model for Data-driven moisture content sensing.

FIG. 3A shows a real part impedance spectroscopy of representative MCC powder; FIG. 3b shows imaginary part impedance spectroscopy of representative MCC powder; FIG. 3C shows RMSD sensing index vs. Moisture content at the frequency of 100-1000 kHz; FIG. 3D shows Heat map of the correlation coefficient between moisture content and the spectroscopy at different frequency.

FIG. 4 shows a schematic of the moisture sensing theory.

FIG. 5A shows the Nyquist plot of the representative MCC powder at different moisture content; FIG. 5B shows the modeling Nyquist plot of the representative MCC powder at different moisture content; FIG. 5C shows representative equivalent circuit model fitting performance of the MCC powder at the moisture content of 7.33%; FIG. 5D shows correlation coefficient between the EIS sensing indices and moisture content of various powders. Here, R_index represents the resistance parameter derived from the parallel resistor in the equivalent circuit model fitting. Q_index, corresponds to the charge parameter from the Constant Phase Element (CPE), and α_index denotes the phase parameter of the CPE component.

FIG. 6A shows real moisture content vs. predicted moisture content from the entire dataset; FIG. 6B shows real moisture content vs. predicted moisture content from the validation dataset.

FIG. 7 Comparative Performance Analysis of Moisture Content Prediction Models. The bar charts display R² values and mean absolute errors (MAE) for various models, highlighting the enhanced accuracy and correlation of the 1DCNN with baseline mechanism (indicated by the red outlines) over other tested algorithms.

FIG. 8 Validation Performance on Different Material Types. The bar chart compares the mean absolute error percentages of the proposed model with and without the baseline across various materials, highlighting the improved accuracy when the baseline is employed.

FIG. 9 shows an experimental Nyquist plot for the pharmaceutical sample microcrystalline cellulose at various moisture content.

FIGS. 10A-D show Nyquist plots through equivalent circuit model for pharmaceutical samples at various moisture content. (FIG. 10A) Microcrystalline Cellulose, (FIG. 10B) Aspirin, (FIG. 10C) Polyvinyl alcohol, and (FIG. 10D) Mannitol.

FIGS. 11A-D show RMSD sensing index from statistical analysis vs. Moisture content at the frequency of 100-1000 kHz for pharmaceutical samples at various moisture content (FIG. 11A) Microcrystalline Cellulose, (FIG. 11B) Aspirin, (FIG. 11C) Polyvinyl alcohol, and (FIG. 11D) Mannitol.

FIGS. 12A-D show EIS sensing index from equivalent circuit modeling for pharmaceutical samples at various moisture content (FIG. 12A) Microcrystalline Cellulose, (FIG. 12B) Aspirin, (FIG. 12C) Polyvinyl alcohol, and (FIG. 12D) Mannitol.

FIG. 13 shows particle size distribution analysis of different testing powders.

FIGS. 14A-B show Correlation coefficient heat map between the input features and output labels for the machine learning task. (FIG. 14A) PCA dimension reduced real part impedance spectroscopy with other features; (FIG. 14B) PCA dimension reduced imaginary part impedance spectroscopy with other features.

FIG. 15 shows learning curve for the moisture content sensing by using proposed 1DCNN model.

FIG. 16 is a high-level diagram showing the components of an exemplary data-processing system.

### DETAILED DESCRIPTION

The invention generally relates to systems and methods for machine learning guided electrochemical impedance spectroscopy for calibration-free pharmaceutical moisture content monitoring. In certain aspects, the invention provides an accurate and efficient method for real-time moisture content monitoring in pharmaceuticals with minimal to zero calibration. Electrochemical impedance spectroscopy (EIS) is utilized herein to measure the electrical properties of samples, such as but not limited to, pharmaceutical materials, under various moisture conditions. EIS effectively discerns the physical properties from electrical characteristics, revealing the intricate relationship between signal behavior and real-time physical characteristics of the samples. Initially, a statistical analysis explores the correlation between EIS signals and moisture content. Additionally, electrochemical simulation sheds light on the underlying mechanism of moisture monitoring via EIS signals.

To enhance the accuracy and effectiveness of moisture content monitoring, we propose the utilization of machine learning techniques, 1D Convolutional Neural Network (1DCNN) for signal processing and the development of a predictive model. The choice of 1DCNN is driven by its excellence in handling time-series data and extracting key features from complex signals, ideal for EIS data interpretation. In the machine learning process, we introduce an innovative concept called the baseline mechanism, which enables the model to learn the chemical, physical, moisture content, and electrical information contained in the signals. By incorporating these multiple dimensions of information, the model can accurately predict the moisture content of pharmaceutical samples, achieving a calibration-free, direct measurement that is adaptable to various sample types and conditions.

It is believed that the systems and methods herein surpass the constraints of current methodologies by introducing a novel, efficient, and calibration-free approach for real-time moisture content monitoring in samples, such as pharmaceutical samples for pharmaceutical manufacturing. With the potential to revolutionize process control and product quality assurance, the invention herein promises a substantial enhancement in the pharmaceutical industry's efficiency and reliability.

### Sample Preparation

As shown herein, four common pharmaceutical raw material powders were selected for testing, including MCC (Microcrystalline Cellulose from AVICEL PH-200), ASP(Aspirin, from Sigma-Aldrich), PVA (PVA, Mw 89,000-98,000, 99+% hydrolyzed, from Sigma-Aldrich), and MA(Mannitol, PEARLITOL 160 C mannitol, from Roquette). To alter the moisture content of these materials, the samples were mixed with deionized water and subsequently placed in separate chambers under different humidity conditions. The humidity levels varied over a substantial range, from 0% to 15%. Once the samples were adequately prepared, they were subjected to further testing under varying humidity conditions.

### Measurement of Moisture Content

To determine the moisture content of the samples, we used the Mettler Toledo HE73 Moisture Analyzer, serving as our reference method for validation. After adjusting the samples to various moisture levels, they were sampled and analyzed using this moisture analyzer. For accuracy, each sample comprised at least 1 gram of the respective pharmaceutical raw material powder. The moisture analyzer operates by employing a heating process to evaporate moisture content from the samples. This process involves heating the samples to 95° C. and maintaining this temperature until a constant weight is achieved, indicating complete water evaporation. The final moisture content is calculated based on the weight difference pre- and post-heating. The moisture content values obtained from this reference method were then used to validate and compare the results of our proposed sensing technique.

### Measurement of Electrical Sensing Signal

The electrical response of the samples was simultaneously measured alongside their moisture content using the Keysight Technologies E4990A impedance analyzer. For these measurements, a constant excitation voltage of 1V, representing the peak amplitude of the AC sine perturbation signal, was applied, with no DC potential bias. The impedance analyzer spanned a frequency range from 20 Hz to 1 MHz, and each spectroscopy measurement generated 1601 data points, uniformly sampled using a linear approach.

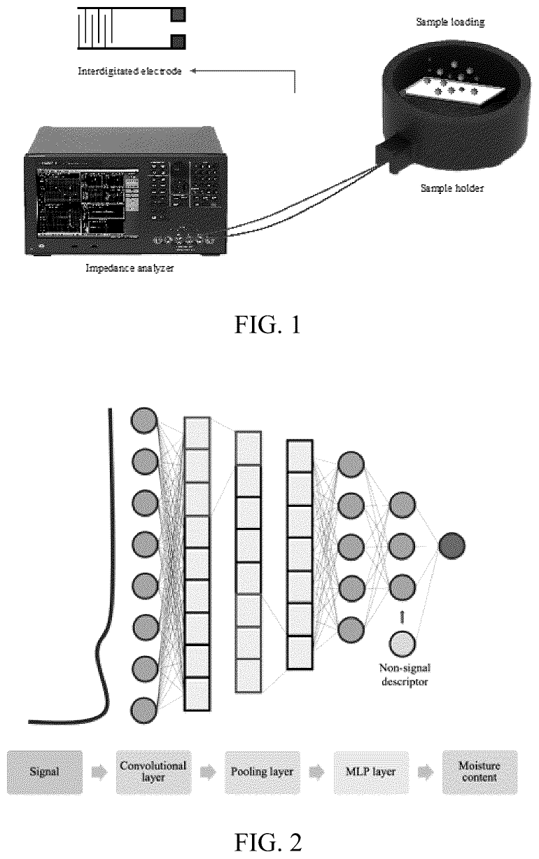

For the sensing setup, we employed commercial Interdigitated Gold Electrodes with specific characteristics to ensure precise signal capture. These electrodes had an overall size of 10 mm×10 mm, with each line being 100 μm wide and spaced 100 μm apart. The metal layers consisted of Cu/Ni/Au with thicknesses of 12 μm, 1 μm, and 1 μm, respectively, laid upon a Polyimide (PI) substrate that was 13 μm thick. To facilitate the measurements, a custom 3D-printed holder was used to mount the samples, with the interdigitated electrodes installed on the holder's back to make contact with the samples and collect signals efficiently. Furthermore, these interdigitated electrodes have proven to be highly compatible with pharmaceutical materials, offering stable signal measurement, repeatable signal output, and enhanced sensitivity to the physical properties of pharmaceuticals. These configurations are illustrated in FIG. 1.

### Signal Processing

After collecting the electrical signal and moisture content data of the samples, a statistical approach was employed to assess the variations in the electrical signals at different moisture levels compared to the signals obtained under dry conditions. The root-mean-square deviation (RMSD)[37,38] was utilized as a statistical index for this comparison. The equation of RMSD sensing index is shown below:

$$RMSD ( \% ) = \sqrt{\frac{{\sum }_{i = 1}^{N} {( {G}_{i} - {G}_{bl} )}^{2}}{{\sum }_{i = 1}^{N} {( {G}_{bl} )}^{2}}} \qquad ( 1 )$$

where Gᵢ is the spectra of sample of various moisture level, G_{bl} is the baseline spectra obtained at the dry stage, Nis the data points in the spectra, respectively.

In addition to the statistical analysis, the study also employed EIS and equivalent circuit modeling to analyze the signals. The equivalent circuit consisted of a series resistor (Rₛ), a parallel resistor (Rₚ), and a parallel constant phase element

$$( CPE , {Z}_{CPE} = \frac{1}{Q * {( j ω )}^{α}} ,$$

Q is the magnitude of the CPE, ω is the angular frequency, j is the unit of imaginary numbers, a is the phase constant, with its value ranging between 0 and 1). After the fitting process, EIS indices, R_index(represents the resistance parameter derived from the parallel resistor in the equivalent circuit model fitting, herein referred as R_index), Q_index(corresponds to the charge parameter from the CPE, herein referred as Q_index), and α_index (denotes the phase parameter of the CPE component, herein referred as α_index) were calculated based on the equivalent circuit parameters obtained from the model. Thus, the measured signal can be expressed by the equation below:

$$Z = {Z}^{′} + {Z}^{″} = {R}_{s} + \frac{1}{\frac{1}{{Z}_{CPE}} + {R}_{p}} \qquad ( 1 )$$

where Z′ is the real part of the spectroscopy, Z″ is the imaginary part of the spectroscopy. The calculated EIS indices were then utilized to establish a correlation with the moisture content of pharmaceutical samples, aiming to elucidate the mechanism by which EIS signals can sense moisture content.

### Moisture Content Sensing Model Developing by Using 1DCNN Algorithm

An innovative approach is provided here, which utilizes a 1DCNN model to establish a moisture content evaluation model, as illustrated in FIG. 2. The 1DCNN model has been widely used for processing spectrum-type signals and has shown promising results in various signal analysis tasks. The 1DCNN model is designed to take the input signals from the samples. These signals are fed into the model through two channels: one containing the real-time moisture content signal of the samples and the other containing the baseline signal of the samples under dry conditions. This approach employs a baseline mechanism to better provide the model with the initial state of the samples, thereby enriching the information available for analysis. The process starts with the 1D convolutional layers, which learn and extract features from the input signals. Subsequently, pooling layers are applied to reduce the dimensionality and highlight the salient features of the signals. After passing through the flatten layer, the processed signals are combined with non-signal descriptors in the Multi-Layer Perceptron (MLP) layer. Detailed information regarding the model's parameters, training process, and comparative analysis of model performance is provided in the Examples herein.

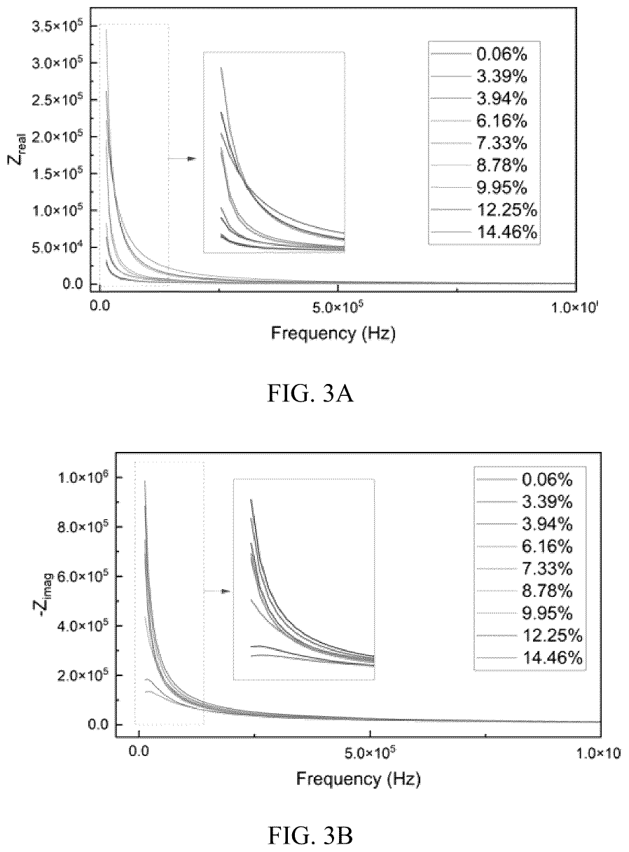

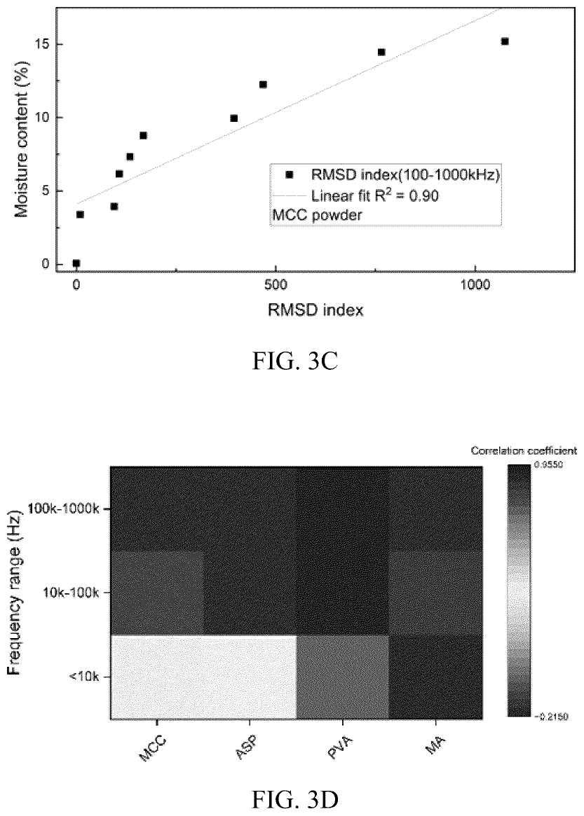

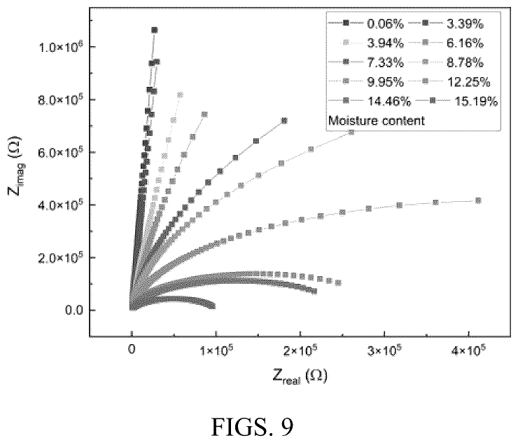

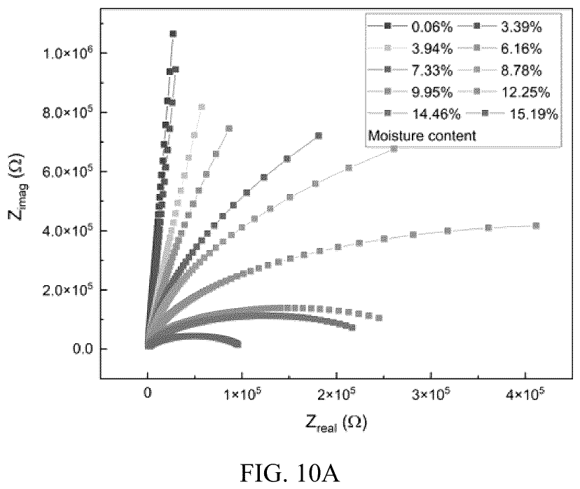

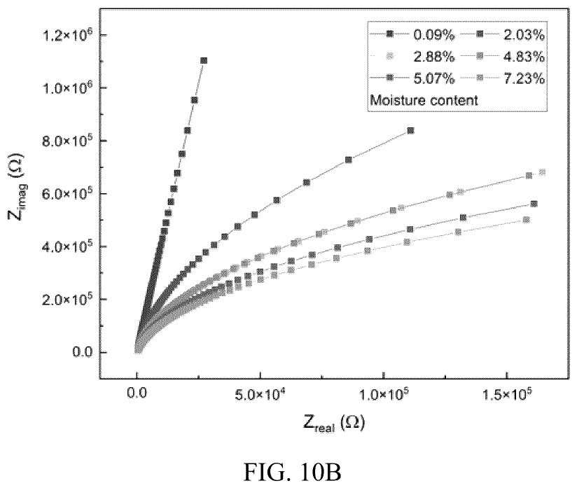

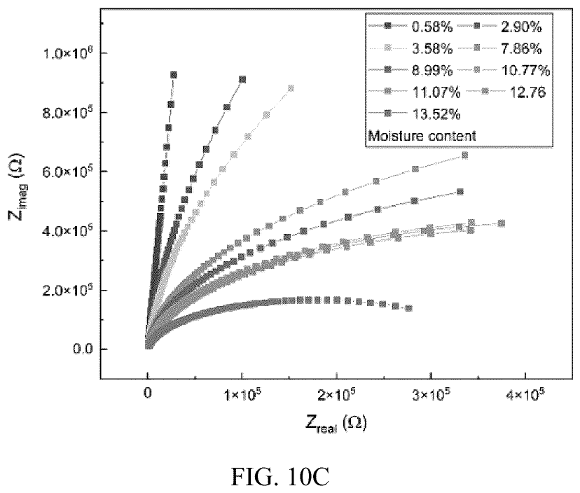

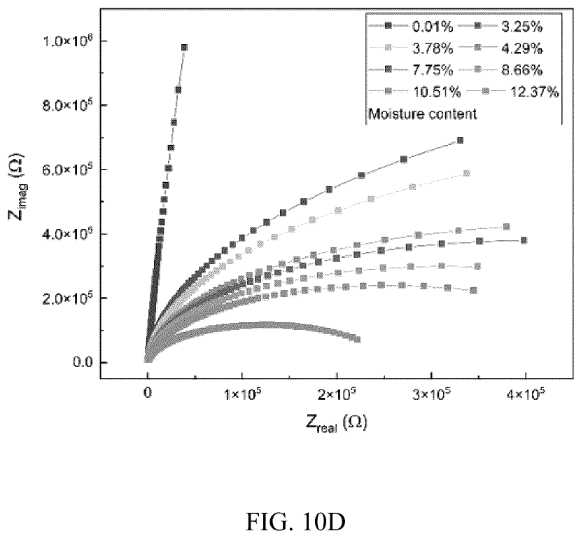

Impedance spectroscopy analysis FIGS. 3A-D demonstrate the changes in electrical signals of MCC powder as a representative material in response to varying moisture contents. It serves as an illustrative example, and additional data is shown in the Examples in FIG. 9 and FIGS. 10A-D. An observable trend is observed where the real part of the EIS signal increases with an augmentation in moisture content. This phenomenon indicates that the electrical properties of the material system within the sample are significantly influenced by changes in moisture content, which is clearly reflected in the EIS measurements. FIG. 3B presents the changes in the imaginary part of the EIS signal, demonstrating a distinct response corresponding to the variation in moisture content. The alterations in both the real and imaginary components of the EIS signal affirm that the spectroscopy of the pharmaceutical material can be effectively employed to track and monitor changes in the moisture content of the samples. The observed sensitivity of the EIS signals to changes in moisture content highlights its potential as an accurate and rapid method for real-time moisture content monitoring in pharmaceutical production processes.

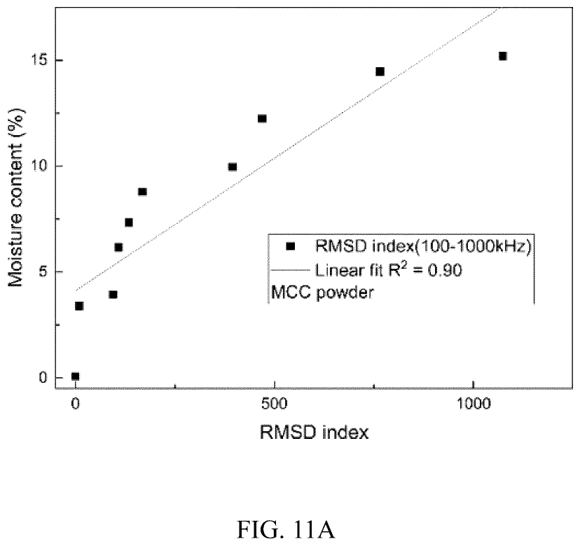

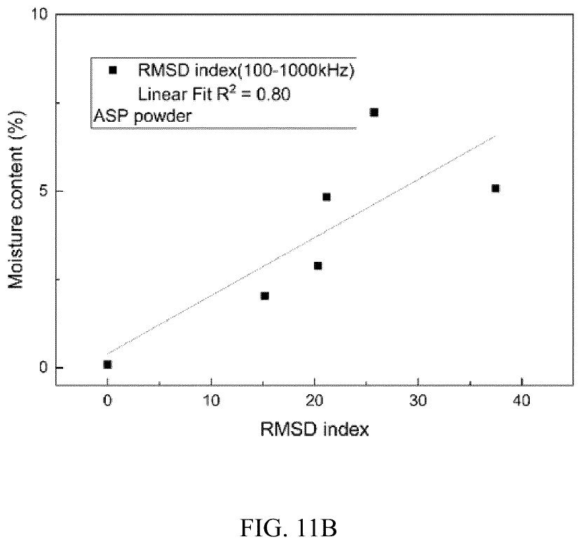

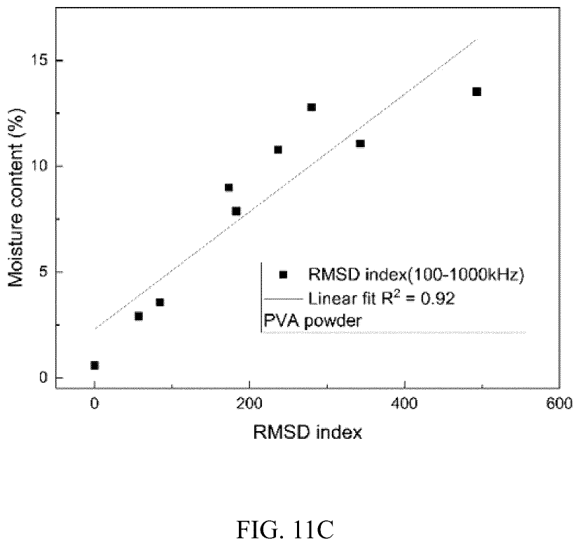

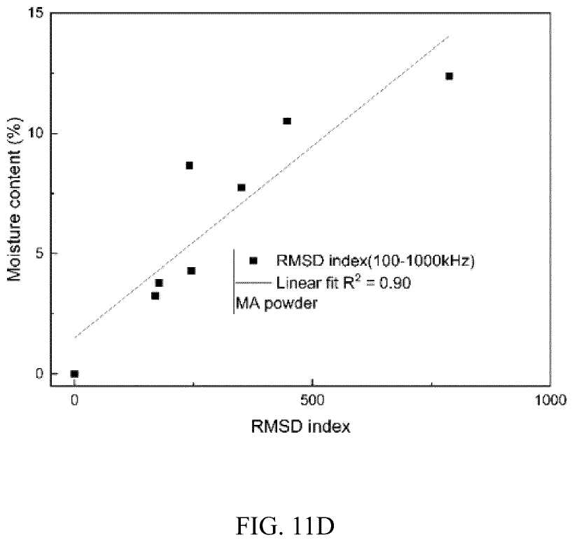

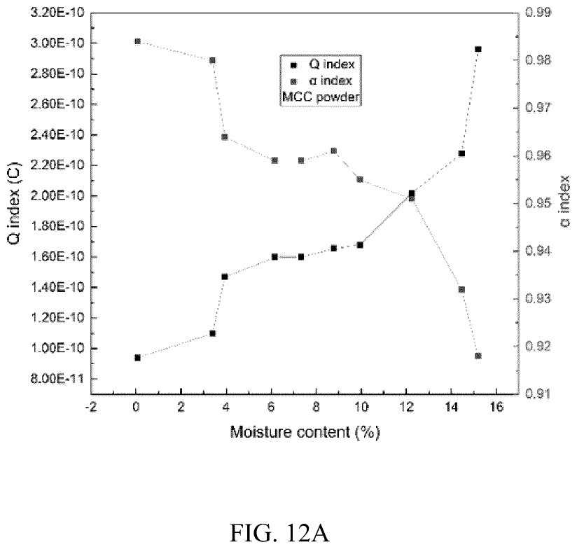

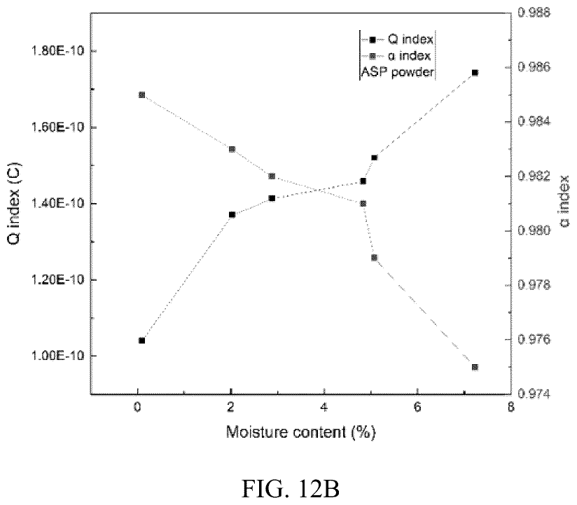

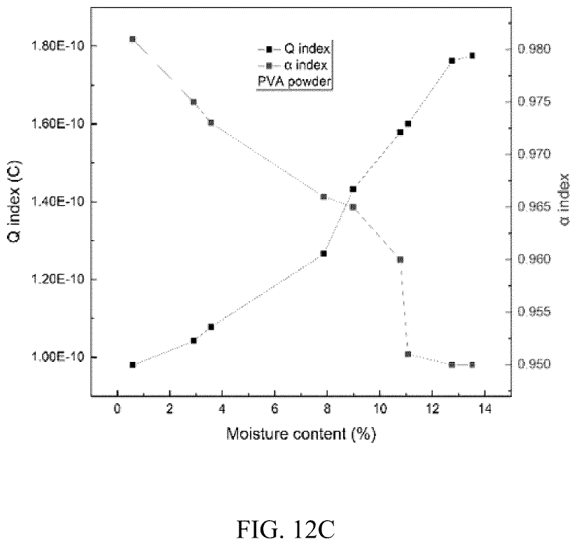

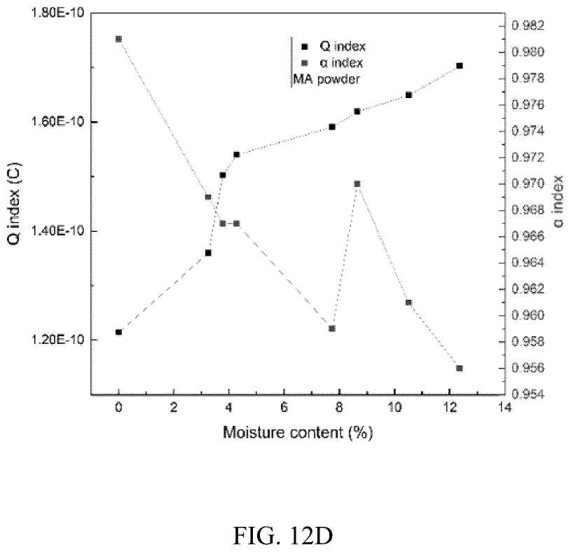

To quantitatively characterize the relationship between signal changes and moisture content, the RMSD index is employed to assess how the electrical signals of the pharmaceutical material perform under varying moisture content conditions compared to the signals observed under dry conditions. FIG. 3C displays the RMSD index calculated within the frequency range of 100-1000 kHz, demonstrating a gradual increase in the index as the moisture content rises. This observation signifies that the RMSD index effectively captures the differences in the electrical signals corresponding to different moisture content levels. The RMSD index sensing performance for the rest pharmaceutical materials are displayed in FIGS. 11A-D. Furthermore, the RMSD index exhibits a positive correlation with the actual moisture content of the samples, with a correlation coefficient of 0.9. This strong correlation highlights the capability of the RMSD index to accurately represent and quantify the variations in moisture content within the pharmaceutical samples. In FIG. 3D, a comparative analysis of the correlation coefficients between moisture content and the RMSD index for different sample materials is presented across various frequency ranges. Remarkably, the frequency range of 100-1000 kHz consistently exhibits a stable and robust capacity to describe the changes in moisture content within the samples. It can be observed that frequencies above 10 kHz exhibit a more significant relationship between the signal and moisture content. The utilization of the RMSD index provides a quantitative means to evaluate the relationship between electrical signals and moisture content accurately. The strong correlation observed between the RMSD index, and the actual moisture content supports its potential as a key parameter for the development of the predictive model using machine learning techniques.

### Equivalent Circuit Modeling

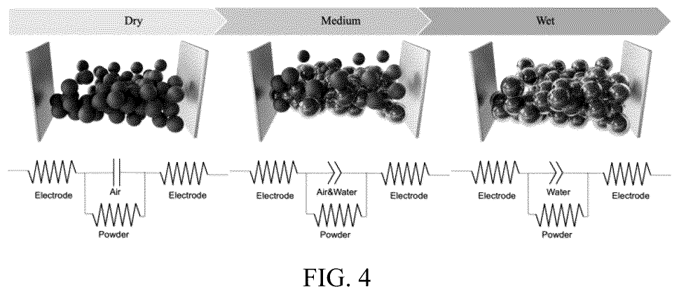

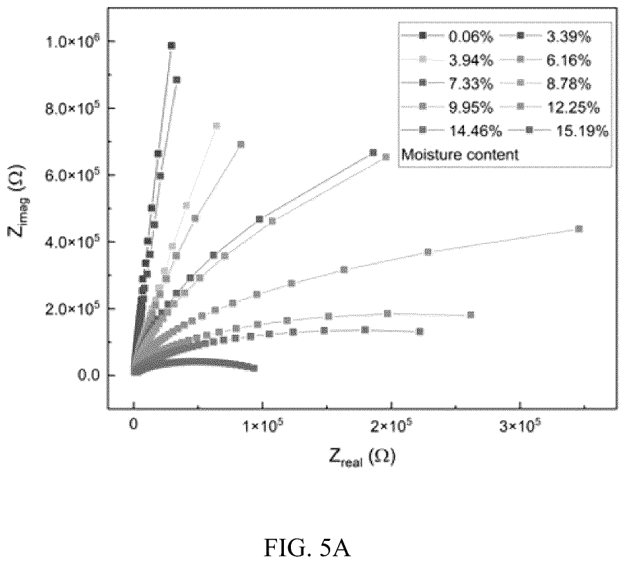

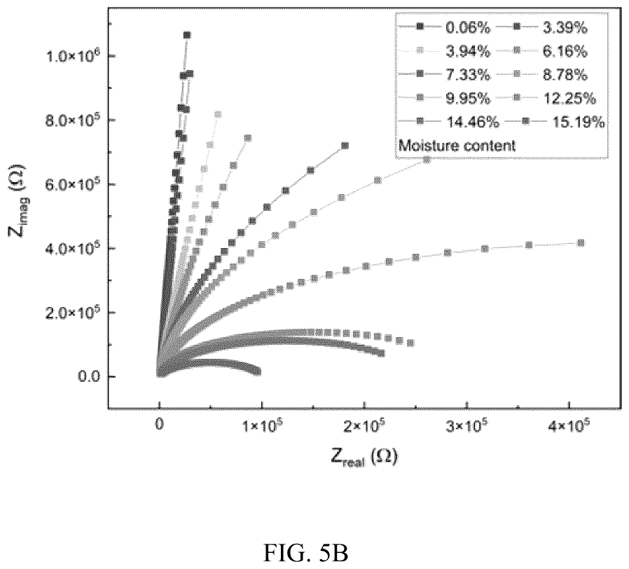

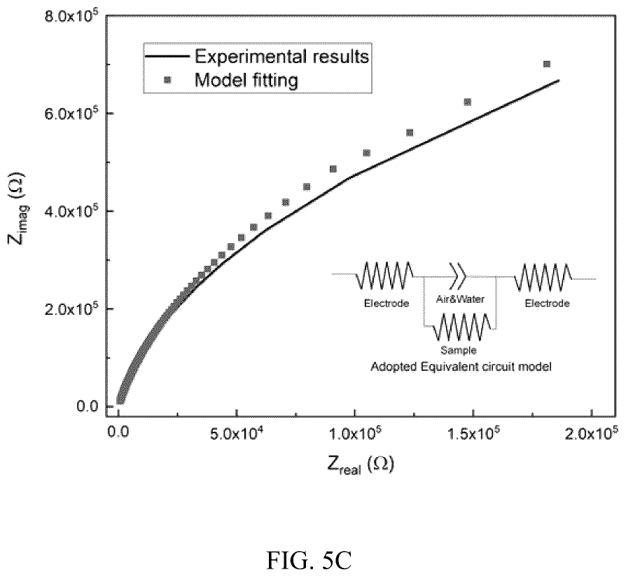

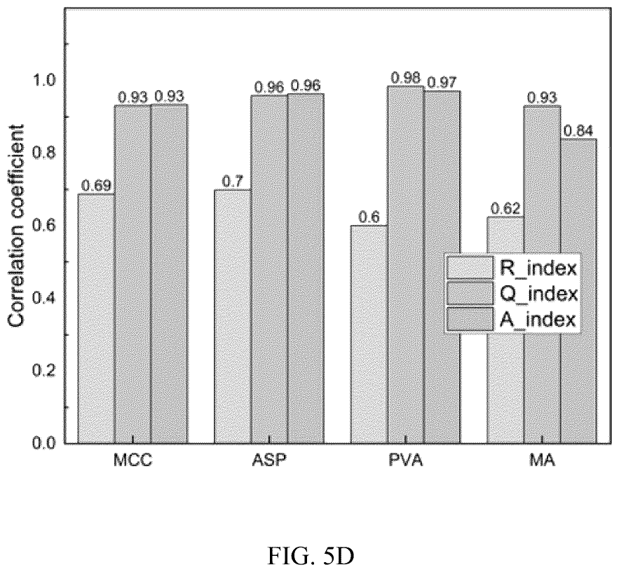

After performing statistical analysis, equivalent circuit fitting was employed to elucidate the relationship between the changes in electrical signals and moisture content. The purpose of equivalent circuit fitting is to utilize electrochemical impedance as a method to reflect the molecular structure and chemical composition of materials through an electrical model. In this context, the sensor and the pharmaceutical materials can be regarded as resistors within the circuit, while the air between the powder particles and the varying moisture content can be considered as capacitors capable of accumulating charge. Due to the presence of different interfaces, the constant phase element (CPE) was selected as a replacement for the traditional capacitor in the circuit simulation. We discovered that the modeling parameters Q_index and α_index from the from the Constant Phase Element (CPE), demonstrate a strong correlation with the moisture content in pharmaceutical samples. Under dry conditions, the electrical response of the sample resembles an equivalent circuit composed of a capacitor and resistor, with the α_index exponent of the CPE component approaching 1. As moisture is introduced, a portion of the sample containing water and air can be represented by an imperfect capacitor using the CPE element. This process is depicted in FIG. 4. To establish a relationship between the electrical signals and moisture content, this study proposes the use of an equivalent circuit as illustrated in FIG. 5A to fit the Nyquist plot data. FIG. 5C represents the fitted Nyquist plot of MCC powder at a moisture content of 7.33%, showing the agreement with the experimental data. This indicates that the proposed equivalent circuit model effectively characterizes the electrical behavior of pharmaceutical samples under different moisture content conditions. FIG. 5A and FIG. 5B compare the fitted data with experimental data using MCC powder as a representative example, demonstrating that the fitted data accurately represents the variations in moisture content within the samples. The fitting performance for the rest 3 types of samples are illustrated in FIG. 9 and FIGS. 10A-D.

It is observed that the Q_index (a parameter representing the accumulation of charge during the testing process) and the α_index (a parameter representing the difference between CPE behavior and a regular capacitor) exhibit high correlation coefficients with the moisture content of the samples. Specifically, the Q_index can be considered to reflect the amount of accumulated charge during the testing process. As the moisture content increases, the presence of water, which can accumulate more charge than air, leads to higher Q_index values, thereby reflecting the moisture content of the samples. On the other hand, the α_index indicates the deviation of the CPE element from a regular capacitor. As the moisture content increases, the CPE behaves less like a normal capacitor, resulting in a decrease in the α_index. The moisture content sensing performance for other pharmaceutical materials can be found in the supporting document, Fig. s4. By analyzing the Q_index and α_index derived from the equivalent circuit fitting, it becomes possible to estimate the moisture content in pharmaceutical materials based on their electrical responses. The high correlation observed between these indices and moisture content further validates the effectiveness of the proposed calibration-free approach for real-time moisture content monitoring in pharmaceutical production processes.

### 1DCNN-Based Moisture Content Sensing Model

In certain of the embodiments herein, Electrochemical Impedance Spectroscopy (EIS) signals, particle size data, and equivalent circuit model fitting parameters were utilized as input features to develop our 1DCNN model for moisture content estimation in pharmaceutical materials. The detailed installation of the input features can be found in the Examples. Our preliminary analysis regarding the input features, detailed in the Examples, indicated a good correlation between these selected features and the moisture content, yet it was clear that a more sophisticated processing technique could unveil deeper insights.

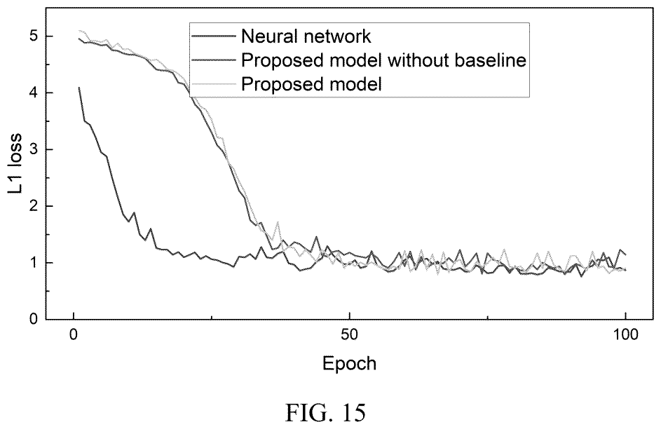

The dataset used in the training process consisted of EIS signals from four different pharmaceutical materials with varying particle sizes and moisture contents. Prior to training, the dataset was divided into training and validation sets to ensure robust model performance. The 1DCNN model initially separates the baseline signal (corresponding to the sample under dry conditions) and the real-time signals (corresponding to the sample under different humidity conditions) into two layers and feeds them into the convolutional layers. Through the convolution, pooling, and flattening processes, the signals are processed and then combined with the physical information of the samples in the fully connected layer. This integration of electrical and physical information enables the model to capture the complex relationship between the input signals and moisture content. During the training process, the model gradually converged after approximately 100 epochs, as shown in FIG. 15. The convolutional and pooling layers play a crucial role in extracting relevant information and reducing the dimensionality of the high-dimensional EIS signals. This allows for effective fusion of the high-dimensional signals with other physical information, providing a conducive environment for the model to better understand the input information and evaluate the moisture content accurately.

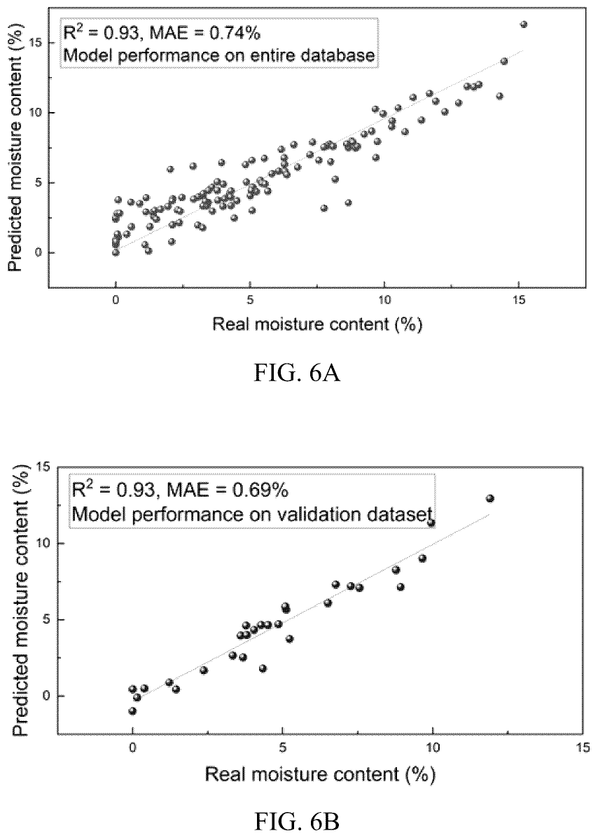

FIGS. 6A-B illustrate the performance of the trained model on the full training set and the validation set, respectively, including all different types of samples. The model achieves an error of 0.69% on the validation dataset. This demonstrates the potential of using machine learning techniques to predict moisture content based on material's electrochemical signals. In future studies, the inclusion of more data and further model adjustments will further enhance the reliability of the model. By integrating both the electrical and physical information in the model, the proposed approach significantly enhances the accuracy of moisture content prediction. The trained 1DCNN model can effectively analyze the complex relationship between the input signals and moisture content, enabling real-time and reliable monitoring without the need for further calibration.

### Comparative Analysis of Model Performance in Moisture Sensing

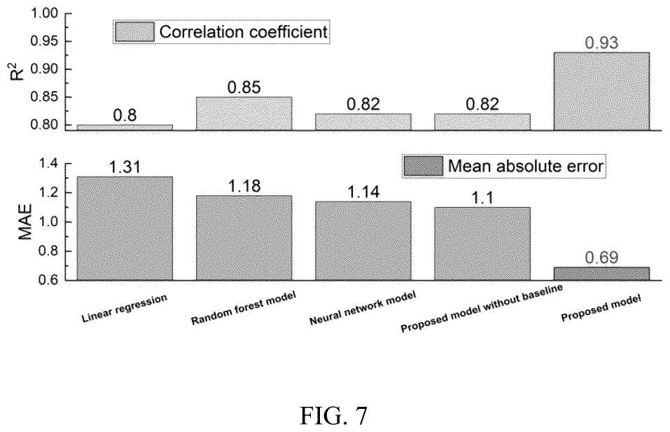

To better illustrate the advantages of our proposed model structure in moisture content sensing and to understand the role of each component, we conducted a comprehensive comparative analysis. FIG. 7 provided succinctly illustrates the performance metrics of various predictive models applied to moisture content estimation in pharmaceutical samples. Linear regression, while foundational, exhibits limited efficacy, as indicated by its lower R2 value and higher mean absolute error (MAE). This suggests that simplistic reliance on EIS indices and statistical measures is inadequate for complex moisture content dynamics. Subsequent implementations using impedance spectrum data to construct random forest regressors and neural networks showed some improvements. However, these models did not reach the high-performance threshold set by the 1DCNN architecture. The lackluster performance of these advanced models, compared to 1DCNN, accentuates the latter's sophisticated signal processing capabilities.

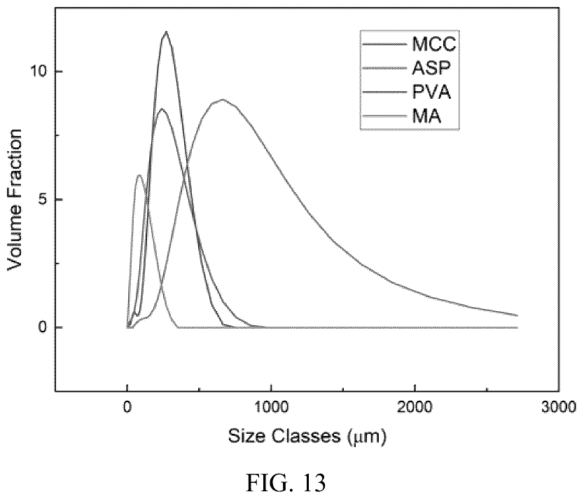

The training loss trends, as depicted in the FIG. 13 from supporting document, provide insight into the learning dynamics of the neural network (NN) and the 1DCNN models. It's noteworthy that the NN model demonstrates a rapid decline in training loss, suggesting an initial quick adaptation to the patterns within the dataset. This could be indicative of the model's simpler architecture, which may allow for faster convergence but potentially at the cost of overfitting or lack of generalizability. Conversely, the 1DCNN exhibits a more gradual reduction in loss over epochs. This slower convergence rate can be attributed to the model's more complex and nuanced structure, which is designed to capture spatial hierarchies in the spectroscopy data. The convolutional layers in the 1DCNN model process the data through multiple filters, which may initially slow down the learning process as the model begins to understand and learn the more intricate patterns in the frequency spectrum of the signals.

The pinnacle of accuracy is achieved with the introduction of the baseline mechanism within the 1DCNN structure, significantly enhancing the model's precision, as reflected by the highest R2 (0.93) value and the lowest MAE (0.69%) in the figure. This baseline channel, representing dry sample conditions, provides a reference point for the model, capturing intrinsic signal characteristics associated with different moisture levels. The incorporation of a baseline mechanism constitutes a paradigm shift, facilitating the 1DCNN's contextual processing of signals by differentiating between fluctuations attributable to moisture content and inherent baseline characteristics. Through extensive and methodical training, the 1DCNN acquires the ability to discern and decode intricate patterns within the data, establishing direct associations with the precise moisture content values. The empirical data illustrated in the figure corroborates that this bifurcated channel strategy not only bolsters the efficiency of the learning process but also significantly enhances the predictive precision of the model.

### Material-Specific Cross-Validation: Enhancing Predictive Generalization

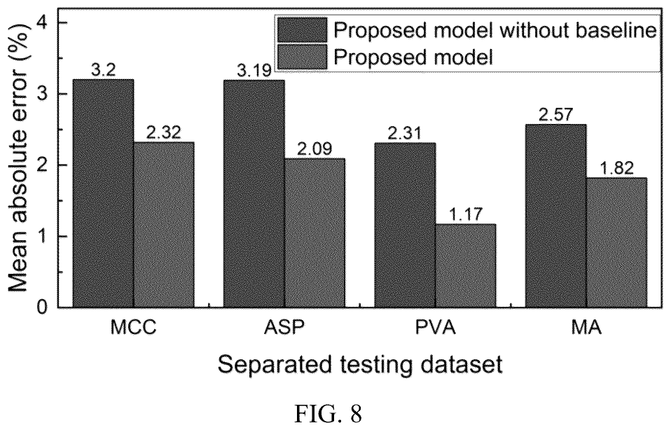

In a departure from conventional random data splitting, certain embodiments herein implemented a methodical cross-validation technique predicated on the intentional separation of data based on material types. This strategy was designed to rigorously test the model's capacity to apply acquired knowledge for the inference of moisture content in novel materials. The performance metrics on the validation material datasets, as depicted in the FIG. 8, are indicative of the model's generalization abilities. Remarkably, the results reveal that the inclusion of the baseline enhances the model's accuracy across different materials. The baseline provides the model with a reference to the initial, unaltered state of the materials, which is instrumental in distinguishing the precise moisture differentials. Consequently, this context-enriched analysis enables the model to make more informed decisions regarding moisture. This is a testament to the baseline's role in bolstering the model's interpretative framework, allowing it to effectively utilize the baseline characteristics as a comparative foundation for accurate moisture determination.

This nuanced approach to cross-validation not only underscores the robustness of the 1DCNN model but also confirms its potential for broader application in scenarios where knowledge transfer to new material types is crucial. The success of this methodology paves the way for the adoption of calibration-free and efficient testing technologies within pharmaceutical production lines. It validates the model's proficiency in leveraging the inherent knowledge embedded within the EIS signals, marking a significant advancement in the domain of predictive moisture content analysis. Such calibration-free methods promise to streamline the analytical process, reduce downtime, and enhance the overall efficiency of pharmaceutical manufacturing, thereby ensuring consistent product quality and compliance with stringent regulatory standards.

### CONCLUSION

As shown herein, the potential of impedance spectroscopy as a reliable method for characterizing moisture content in pharmaceutical materials has been demonstrated. Through the utilization of impedance signals, we successfully investigated the correlation between electrical properties and varying moisture contents in pharmaceutical samples.

Through the utilization of equivalent circuit modeling, we successfully interpreted the mechanism underlying impedance spectroscopy's sensitivity to moisture content variations. The equivalent circuit analysis allowed us to identify key parameters which exhibited strong correlations with the actual moisture content of the samples. These findings highlighted the significance of electrical and physical information in tracking moisture content changes and provided valuable insights into how impedance spectroscopy can effectively characterize moisture content in pharmaceutical materials.

Leveraging AI techniques, a 1DCNN model was developed to process the complex spectroscopy data effectively. By combining the frequency spectrum information, equivalent circuit indices, and material characteristics as inputs, our proposed model achieved remarkable results. The model's predictive capability produced a moisture content prediction model with an average error as low as 0.69%. This exceptional accuracy eliminates the need for calibration, providing a rapid and calibration-free solution for real-time moisture content monitoring in pharmaceutical production.

In summary, the embodiments herein leverage impedance spectroscopy as a robust method for quantifying moisture content in pharmaceutical materials. By unraveling the mechanism through equivalent circuit modeling and harnessing the power of AI, we successfully developed an accurate predictive model. The combination of these techniques represents a significant advancement in real-time moisture content monitoring in the pharmaceutical industry. Furthermore, the methodologies and findings from this study have broader applications in other industries such as food processing, agriculture, and electronics, where moisture content plays a crucial role in product quality and longevity. The adaptability of our approach suggests potential for its integration into various manufacturing and quality control processes, offering a versatile tool for moisture content analysis across diverse sectors.

### System Architecture

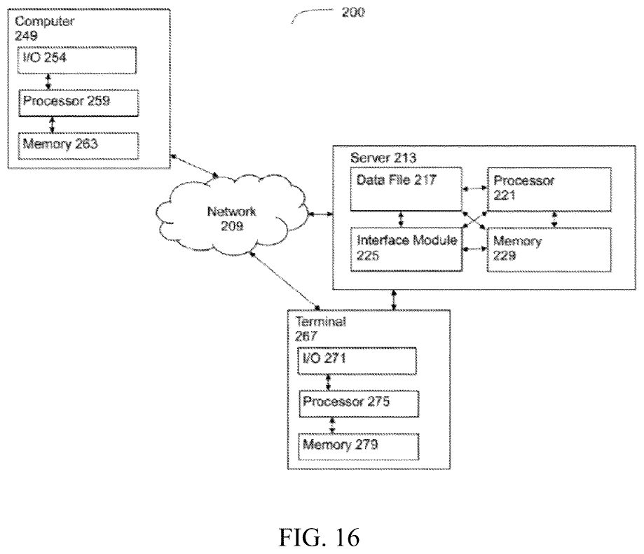

FIG. 16 is a high-level diagram showing the components of an exemplary data-processing system 1000 for analyzing data and performing other analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The data described above may be obtained using detector 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 1086 which in one embodiment may be capable of real-time calculations (and in an alternative embodiment configured to perform calculations on a non-real-time basis and store the results of calculations for use later) can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086.

The peripheral system 1020 can include one or more devices configured to provide digital content records to the processor 1086. For example, the peripheral system 1020 can include medical devices (such as medical imaging devices), digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (e.g., a tablet) connected, e.g., via a network or a null-modem cable, or any device or combination of devices from which data is input to the processor 1086. The user interface system 1030 also can include a display device, a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), Universal Serial Bus (USB) interface memory device, erasable programmable read-only memories (EPROM, EEPROM, or Flash), remotely accessible hard drives, and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors) to cause functions, acts, or operational steps of various aspects herein to be performed by the processor 1086 (or other processor). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: 1DCNN Model Architecture and Parameters

The work herein provides an innovative approach using a 1D Convolutional Neural Network (1DCNN) for water content evaluation, as. The model is specifically designed to process input signals from the pharmaceutical samples in two separate channels: one channel handles the real-time water content signal, and the other manages the baseline signal under dry conditions. This dual-channel approach, incorporating a baseline mechanism, significantly enhances the model's ability to capture the initial state of the samples, enriching the analysis. As shown in Table. S1, the model architecture begins with an input Conv1d layer (Layer 1) that handles inputs to batch size channels with a kernel size of 2. This is followed by a MaxPoolId layer (Layer 2) with a kernel size of 1 for pooling, effectively reducing the dimensionality of the input data. The next stage is another Conv1d layer (Layer 3) that expands the batch size to 32 channels with a kernel size of 1. This layer is crucial for feature extraction from the input signals. The output from this convolutional layer is then flattened (Layer 4) to prepare it for batch normalization. Layer 5 employs BatchNorm1d with 32 features to normalize the output from the Flatten layer, enhancing the stability and efficiency of the model. The data then passes through a Linear layer (Layer 6), transforming from 32 to 16 units. In Layer 7, these 16 units are merged with additional features including one-hot encoded sample type information and particle size information. The one-hot encoding is used to differentiate the various sample types in the study, while the particle size data is acquired using the Malvern Mastersizer 3000 Laser diffraction particle size analyzer (PSA). The final output layer (Layer 8) is a Linear layer that maps these combined features from 18 units to the final output units, which correspond to the moisture content of the samples.

### Example 2: Algorithmic Benchmarking and Model Selection

To rigorously validate our proposed 1D Convolutional Neural Network (1DCNN) model, we initially explored a variety of algorithms for model training. This exploratory phase included testing with linear regression, a machine learning-based random forest regressor, a simple neural network, and a 1DCNN structure without the baseline mechanism. The purpose of this diverse algorithmic testing was to benchmark and highlight the necessity and superiority of our designed model structure. In particular, the inclusion of the baseline in the 1DCNN model emerged as a critical factor for enhanced performance.

### Example 3: Hyperparameter Optimization

In our comprehensive study, we not only optimized the 1DCNN model but also compared it with linear regression, random forest regressor, and a standard neural network. For the 1DCNN model, we selected a batch size of 32 and a learning rate of 1e⁻⁵, guided by the model's performance on the validation set. The L1 loss function was chosen for its precision in error reduction. In contrast, the random forest regressor underwent a grid search optimization, enabling us to fine-tune its parameters for maximal effectiveness. This comparative approach in hyperparameter optimization across different models was instrumental in evaluating the relative strengths and applicability of each algorithm in our study.

### Example 4: Material-Specific Cross-Validation

For cross-validation, we adopted a train-test splitting strategy, allocating 70 percent of the data for training and the remaining 30 percent for validation. This approach enabled us to assess the model's performance on an unseen validation set, ensuring the robustness and generalizability of our findings. Furthermore, we employed a more nuanced method of cross-validation by dividing the data based on the types of samples. This strategy was particularly insightful as it allowed us to test the model's ability to leverage existing knowledge and apply it to predict the moisture content of entirely new, external materials. Such a validation method not only confirms the efficacy of our 1DCNN model with the baseline mechanism but also underscores the versatility and accuracy of electrochemical impedance spectroscopy (EIS) signals in estimating moisture content across varying sample types (FIG. 9) Comparative Performance Analysis of Moisture Content Prediction Models. The bar charts display R² values and mean absolute errors (MAE) for various models, highlighting the enhanced accuracy and correlation of the 1DCNN with baseline mechanism.

TABLE 1
Detailed 1D CNN Model Structure

| No. | Layer Type | Class | Description |
|---|---|---|---|
| 1 | Input | Conv1d | Conv1d layer with inputs |
| | Layer | | to batch size channels and |
| | | | kernel size of 2 |
| 2 | Pooling Layer | MaxPool1d | MaxPool1d with kernel size of 1 |
| 3 | Convolutional | Conv1d | Conv1d layer with batch |
| | Layer | | size to 32 channels and |
| | | | kernel size of 1 |
| 4 | Flatten Layer | Flatten | Flatten the output |
| 5 | Batch | BatchNorm1d | BatchNorm1d with |
| | normalization | | 32 features |
| 6 | Linear Layer | Linear | Linear layer from 32 to 16 units |
| 7 | Additional | Linear | 16 units merge |
| | features | | with additional features |
| 8 | Output | Linear | Linear layer from 18 |
| | Layer | | to outputs units (Moisture |
| | | | content) |

### Example 5: Particle Size Analysis

To enhance the accuracy of moisture content predictions, particle size metrics were included to more comprehensively describe the physical characteristics of the pharmaceutical materials. The impact of particle size, shape, and surface area on the Electrochemical Impedance Spectroscopy (EIS) signal is multifaceted. Specifically, these physical properties influence the extent and nature of contact between water molecules and the material, which in turn affects the dielectric properties and the impedance spectrum. FIG. 13 illustrates the unique particle size profile for each pharmaceutical sample, as evidenced by their respective particle size distribution histograms.

In the realm of EIS, smaller particles typically exhibit a higher surface area to volume ratio, leading to increased capacitance and a noticeable shift in the impedance spectrum. Similarly, particle shape affects the compactness of packing and the porosity of the material matrix, further altering the EIS response. By integrating particle size information into the 1DCNN model as a feature, we provide contextual insights into the sample's physical form, enabling the model to adjust its moisture content estimations accordingly. The inclusion of particle size data thereby assists in isolating the moisture-dependent signals from those influenced by the physical form of the material. This step is crucial in moving towards a calibration-free model, as it reduces the model's reliance on predefined calibrations for each sample type and leads to more accurate, material-agnostic moisture content estimations.

### Example 6: Features Pre-Analysis for 1DCNN Model Training

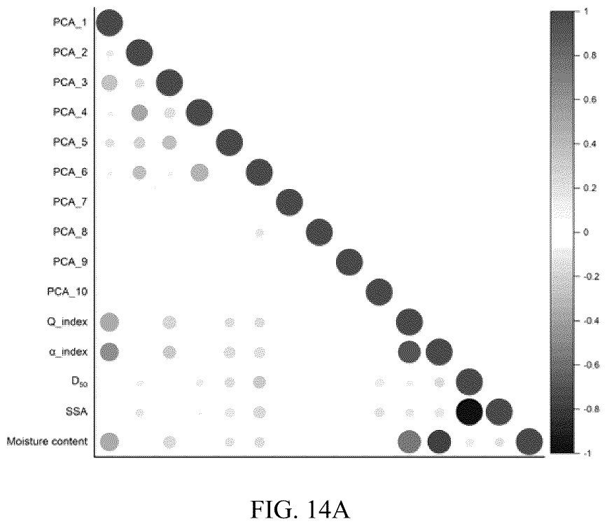

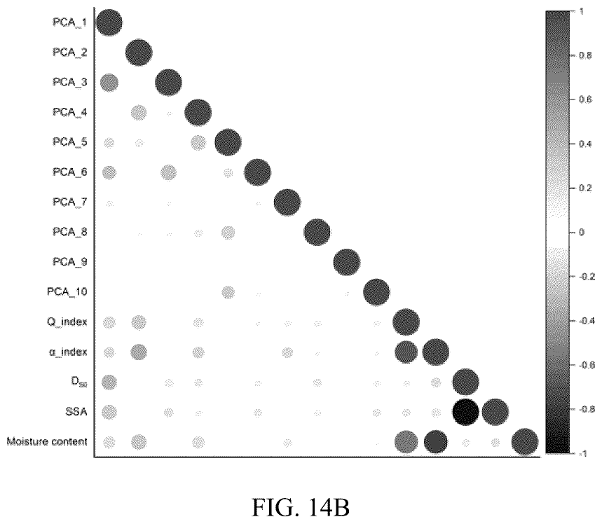

We conducted a pre-analysis of all the features describing the samples to understand the relationship between the model's input and output. Due to the substantial amount of data from the signal type inputs, we employed PCA (Principal Component Analysis) as a dimensionality reduction technique. Specifically, the first ten principal components from both the real and imaginary parts of the impedance spectroscopy data were selected for correlation analysis. Additionally, parameters obtained from the equivalent circuit fitting, namely, Q_index, α_index, D50 (the size in microns at which 50% of the sample is smaller and 50% is larger), and SSA (Specific Surface Area), were included to represent the electrical, chemical, and physical properties of the materials and were also fed into the model as features. The correlation analysis results are illustrated in FIGS. 14A-B, revealing significant correlations between the frequency spectrum information, equivalent circuit indices, and the moisture content of the samples. On the other hand, the input features themselves showed no apparent correlations with moisture content. Therefore, these informative representations were used as inputs to the 1DCNN model, to train the model and create a predictive model for moisture content.

What is claimed is:

1. A system for determining moisture content in a sample, the system comprising:
an electrochemical impedance spectroscopy (EIS) apparatus; and
a processor configured to:
receive electrical properties of a sample from the EIS apparatus; and
correlate the electrical properties to moisture content of the sample.

2. The system of claim 1, wherein the correlate step occurs without calibration.

3. The system of claim 1, wherein the system determines moisture content in real-time.

4. The system of claim 1, wherein the correlate step utilizes a statistical analysis approach that comprises:
obtaining moisture content data of a sample;
obtaining simultaneously electrical response signals of the sample; and
assessing variations in the electrical response signals at different moisture levels compared to electrical response signals obtained under dry conditions for the sample.

5. The system of claim 1, wherein the correlate step utilizes equivalent circuit modeling calculates EIS indices.

6. The system of claim 5, wherein the EIS indices are utilized to establish a correlation with the moisture content of the sample.

7. The system of claim 5, wherein the equivalent circuit modeling comprises:
receive input signals from the sample, which are provided through two channels: a first channel containing the real-time moisture content signal of the sample and a second channel containing baseline signal of the sample under dry conditions.
apply 1D convolutional layers, which learn and extract features from the input signals;
apply pooling layers to reduce dimensionality and highlight salient features of the input signals;
pass through a flatten layer; and
combine with non-signal descriptors in a Multi-Layer Perceptron (MLP) layer.

8. The system of claim 1, wherein the sample is a pharmaceutical sample.

9. The system of claim 1, wherein the processor is integrated into the EIS apparatus.

10. The system of claim 1, wherein the processor is remotely coupled to the EIS apparatus.

11. A method for determining moisture content in a sample, the system comprising:
receiving to a processor electrical properties of a sample from an electrochemical impedance spectroscopy (EIS) apparatus; and
correlating via the processor the electrical properties to moisture content of the sample to thereby determine moisture content in the sample.

12. The method of claim 11, wherein the correlating step occurs without calibration.

13. The method of claim 11, wherein the method determines moisture content in real-time.

14. The method of claim 11, wherein the correlating step utilizes a statistical analysis approach that comprises:
obtaining moisture content data of a sample;
obtaining simultaneously electrical response signals of the sample; and
assessing variations in the electrical response signals at different moisture levels compared to electrical response signals obtained under dry conditions for the sample.

15. The method of claim 11, wherein the correlating step utilizes equivalent circuit modeling calculates EIS indices.

16. The method of claim 15, wherein the EIS indices are utilized to establish a correlation with the moisture content of the sample.

17. The method of claim 15, wherein the equivalent circuit modeling comprises:
receive input signals from the sample, which are provided through two channels: a first channel containing the real-time moisture content signal of the sample and a second channel containing baseline signal of the sample under dry conditions.
apply 1D convolutional layers, which learn and extract features from the input signals;
apply pooling layers to reduce dimensionality and highlight salient features of the input signals;
pass through a flatten layer; and
combine with non-signal descriptors in a Multi-Layer Perceptron (MLP) layer.

18. The method of claim 11, wherein the sample is a pharmaceutical sample.

19. The method of claim 11, wherein the processor is integrated into the EIS apparatus.

20. The method of claim 11, wherein the processor is remotely coupled to the EIS apparatus.
